(19) United States
(12) Patent Application Publication
(10) Pub. No.: US 2019/0209780 A1
(43) Pub. Date: Jul. 11, 2019

(54) AUTOINJECTOR AND USER WEARABLE AUTOINJECTOR ASSEMBLY

(71) Applicant: Blue Lead Medical LLC, Fishers, IN (US)

(72) Inventor: Brendon Mattiuz, Indianapolis, IN (US)

(73) Assignee: Blue Lead Medical LLC, Fishers, IN (US)

(21) Appl. No.: 16/304,746

(22) PCT Filed: May 22, 2017
(86) PCT No.: PCT/US17/33890

§ 371 Date: Nov. 27, 2018

Related U.S. Application Data
(60) Provisional application No. 62/343,117, filed on May 30, 2016.

(51) Int. Cl. A61M 5/20; A61M 5/31; A61M 5/315

(52) U.S. Cl. CPC A61M 5/2033; A61M 5/002; A61M 5/31566; A61M 5/3129

(57) ABSTRACT

An autoinjector includes an elongate housing having a plurality of housing pieces, and foldable about a hinge between a use configuration and a storage configuration. A medicament cartridge is positioned within one of the housing pieces, and an actuator within another one of the housing pieces. A safety is coupled with the actuator and adjusts is between a locking state inhibiting firing the actuator, and a release state, and is positionable in the locking state in each of the use configuration and the storage configuration.

### TECHNICAL FIELD

The present disclosure relates generally to an autoinjector, and more particularly to an autoinjector that is foldable for convenient storage and wearing upon one's person.

### BACKGROUND

Autoinjectors for medicaments are well known and widely used. Many allergy sufferers will be familiar with autoinjectors for delivering a measured dose of potentially life-saving epinephrine. Autoinjectors are also used for other injectable liquids and suspensions, and for users that need a medicament delivered quickly, reliably, and often in a pre-measured dose. The use of autoinjectors has increased worldwide with the rise in prevalence of certain allergies, as well as regulatory changes that have mandated that autoinjectors be kept on hand at schools and other institutional facilities.

An alternative to autoinjectors for at least certain medicaments is the use of a traditional syringe used to load a medicament from a storage vial and manually inject the medicament into the body of a user. Obviously, some skill can be required to successfully obtain a measured dose while maintaining a sterile field in connection with preparing a traditional syringe for medicament delivery. Especially under conditions of duress, loading a syringe from a traditional vial and carrying out a successful injection can be challenging. For these and other reasons, autoinjectors provide advantages over traditional medicament delivery.

U.S. Pat. No. 9,199,037 to Buchine et al. is directed to a portable drug mixing and delivery system and method, and apparently provides an auto-injector for storing wet and dry medicament components separately. Prior to injection the various medicaments within the portable device can be mixed through a multi-stage process. Other autoinjectors are structured for use with a single-component injectable medicament, but suffer from other shortcomings such as being bulky or otherwise difficult to carry, store, or transport. The techniques set forth in Buchine et al. and elsewhere may have certain applications, however, there remains ample room additional and alternative technical developments in the autoinjector field.

### SUMMARY OF THE INVENTION

In one aspect, an autoinjector includes an elongate housing having a first housing piece forming a proximal housing end and defining a first cavity, and a second housing piece forming a distal housing end and defining a second cavity. The autoinjector further includes a hinge coupling together the first housing piece and the second housing piece, and the first housing piece and the second housing piece being rotatable about the hinge to fold the elongate housing between a use configuration where the first cavity and the second cavity are in communication, and a storage configuration. The autoinjector further includes a medicament cartridge positioned within the second cavity, and including a cartridge body, and an injection needle extending from the cartridge body in a direction of the distal housing end. The autoinjector further includes an actuator positioned within the first cavity, and including a force-producing device, and a plunger coupled with a force-producing device. The force-producing device is adjustable from an energized state to a deployed state, to advance the plunger into the second cavity and urge the medicament cartridge in a distal direction to extend the injection needle past the distal housing end.

In another aspect, an autoinjector includes an elongate housing having a plurality of housing pieces and a hinge coupling together two of the housing pieces. The two of the housing pieces are rotatable about the hinge to fold the autoinjector between a use configuration, and a storage configuration. The autoinjector further includes a medicament cartridge positioned within the elongate housing and including an injection needle, and an actuator for deploying the injection needle, positioned within the elongate housing. The autoinjector further includes a safety coupled with the actuator, and adjustable between a locking state at which the safety inhibits firing the actuator, and a release state, and the safety being positionable in the locking state in each of the use configuration and the storage configuration of the autoinjector.

In still another aspect, an autoinjector assembly includes an autoinjector having an elongate housing with a first housing piece, and a second housing piece, and a hinge coupling together the first housing piece and the second housing piece. The first housing piece and the second housing piece are rotatable about the hinge to fold the elongate housing between a use configuration, and a storage configuration. The autoinjector further includes a medicament cartridge including an injection needle, and an actuator for advancing the medicament cartridge through the elongate housing to extend the injection needle out of the elongate housing. The autoinjector assembly further includes a carrying accessory having a container with a cavity formed therein and receiving the autoinjector in the storage configuration, and a strap attached to the container and structured for wearing of the carrying accessory by a user.

### BRIEF DESCRIPTION OF THE DRAWINGS

FIG. 1 is a diagrammatic view of an autoinjector assembly, according to one embodiment:

FIG. 2 is a side diagrammatic view of an autoinjector according to one embodiment, in a storage configuration;

FIG. 3 is a side diagrammatic view of the autoinjector of FIG. 2, in a use configuration:

FIG. 4 is a sectioned side diagrammatic view of the autoinjector of FIGS. 2 and 3, in a storage configuration;

FIG. 5 is a disassembled view of the autoinjector of FIGS. 2-4;

FIG. 6 is a sectioned side diagrammatic view of an autoinjector shown as it might appear positioned against the body of a user and ready for firing;

FIG. 7 is a view similar to FIG. 6 showing the autoinjector having been fired, with an injection needle extended into the body of a user;

FIG. 8 is a view similar to FIGS. 6 and 7, where the autoinjector is fully fired and medicament has been injected into the body of the user;

FIG. 9 is a sectioned side diagrammatic view of a portion of an autoinjector, according to one embodiment;

FIG. 10 is a partial sectioned side diagrammatic view of an autoinjector, according to another embodiment, in one configuration;

FIG. 11 is a sectioned side diagrammatic view of the autoinjector of FIG. 10, in another configuration;

FIG. 12 is a side diagrammatic view of an autoinjector according to yet another embodiment, in one configuration;

FIG. 13 is a side diagrammatic view of an autoinjector as in FIG. 12, in another configuration;

FIG. 14 is a disassembled view of an autoinjector, according to yet another embodiment;

FIG. 15 is an end view of the autoinjector of FIG. 14, in a storage configuration:

FIG. 16 is a sectioned side diagrammatic view of the autoinjector of FIGS. 14 and 15; and

FIG. 17 is a sectioned side diagrammatic view of the autoinjector of FIGS. 14-16, shown as it might appear having been fired.

### DETAILED DESCRIPTION

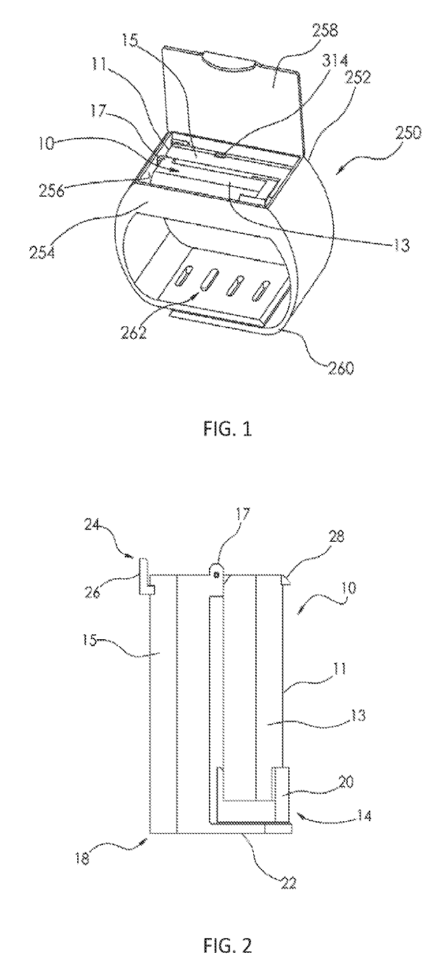

Referring to FIG. 1, there is shown an autoinjector assembly 250, according to one embodiment. Autoinjector assembly 250 is structured for easy carrying, wearing, transport, or storage by a user. As discussed above, autoinjectors of known designs can be bulky, or otherwise difficult to conveniently store on one's person. As will be further apparent from the following description, autoinjector assembly 250 includes an autoinjector 10 and a carrying accessory 252 that cooperate to provide a convenient and efficient package expected to have various advantages over known designs.

To this end, autoinjector 10 includes an elongate housing 11 having a first housing piece 13, and a second housing piece 15, and a hinge 17 coupling together first housing piece 13 and second housing piece 15. First housing piece 13 and second housing piece 15 are rotatable about hinge 17 to fold elongate housing 11 and thus autoinjector 10 between a use configuration, and a storage configuration. In the illustrated embodiment, first housing piece 13 and second housing piece 15 are rotatable relative to one another about an angle that is equal to about 180 degrees. Also in the illustrated embodiment, elongate housing 11 includes a plurality of housing pieces numbering two. In other embodiments, a number of housing pieces greater than two, foldable about a number of hinges greater than one, or foldable about an angle greater than 180 degrees or less than 180 degrees, might be used. A viewing window 314 in housing piece 15 can include either a through-hole whereby a user can visually inspect the contents, such as to determine discoloration, crystallization, vial breakage or cracking, or a transparent covering.

Carrying accessory 252 includes a container 254 having a cavity 256 formed therein and receiving autoinjector 10 in the storage configuration. Container 254 could include a molded plastic body including a lid 258 structured to open and close to seal or block cavity 256 and contain autoinjector 10 therein. Autoinjector 10 could also be retained amongst prongs (not shown), with hook and loop-style fastening such as Velcro®, slid into a sleeve, held in place with a strap or the like, or retained within or upon container 254 in any other suitable manner. In a practical implementation, autoinjector 10 has a substantially rectangular shape in the storage configuration, and cavity 256 has a substantially rectangular shape congruent with the substantially rectangular shape of autoinjector 10. It can further be noted that autoinjector assembly 250 has a form generally analogous to a wristwatch. A strap 260 is attached to or formed integrally with container 254, and can form a band or loop about a user's wrist, ankle, belt loop, et cetera, and otherwise engaging carrying accessory 252 about the body of a user or the user's accoutrements. Strap 260 might be suited for wearing carrying accessory 252 as a necklace or a keychain. A clasp 262 that could include a hook-and-loop fastener clasp, a buckle, snaps, hooks, interference fitting buttons, or any other suitable structure capable of clasping together two parts of strap 260 may be further provided.

As noted above, shape and functionality of autoinjector 10 renders autoinjector 10 well-suited for storage, and provides a relatively low volumetric footprint in three-dimensional space. Referring also now to FIG. 2, there is shown autoinjector 10 as it might appear in the folded, storage configuration. First housing piece 13 and second housing piece 15 each have elongate shapes and are arranged generally such that longitudinal axes (not shown) of the respective first housing piece 13 and second housing piece 15 would be oriented parallel to one another in the storage configuration. Hinge 17 couples first housing piece 13 to second housing piece 15, and will be understood to define a transverse hinge axis that is oriented generally perpendicular to the longitudinal orientations or axes of first housing piece 13 and second housing piece 15. A latch 24 is provided, including a first component 26 upon second housing piece 15, and a second component 28 that is structured to releasably engage with first component 26 attached to first housing piece 13. As will be further apparent from subsequently discussed illustrations, latch 24 can fix elongate housing 11 and autoinjector 10 in the use configuration at which first housing piece 13 and second housing piece 15 are generally in coaxial, end-to-end alignment. First housing piece 13 forms a proximal housing end 14, and second housing piece 15 forms a distal housing end 18. In the illustrated embodiment, a finger guard 22 extends radially outward from distal housing end 18 and a safety 20 is positioned upon proximal housing end 14. Finger guard 22 holds safety 20 captive between proximal housing end 14 and finger guard 22 in the storage configuration depicted in FIG. 2. As further discussed herein, safety 20 inhibits firing an actuator within autoinjector 10. It can also be seen from FIG. 2 that safety 20 is coupled with first housing piece 13 and caps proximal housing end 14.

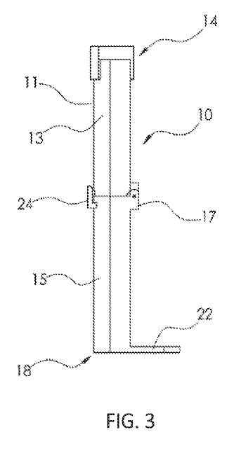

Referring also now to FIG. 3, there is shown autoinjector 10 as it might appear folded from the storage configuration to the use configuration, and illustrating latch 24 in a latched state, with first housing piece 13 and second housing piece 15 arranged generally end-to-end. It can be seen that first housing piece 13 and second housing piece 15 would be generally understood to be in coaxial, end-to-end alignment. In other embodiments, the respective housing pieces might not be arranged coaxially in the use configuration, nor arranged end to end as such.

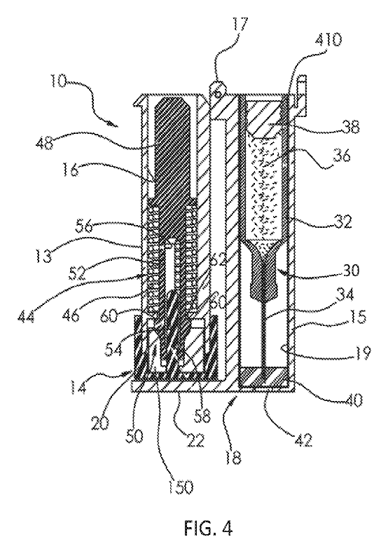

Referring also now to FIG. 4, there is shown a sectioned view through autoinjector 10 as it might appear in the storage configuration, and flipped over relative to the illustration shown in FIG. 2. First housing piece 13 defines a first cavity 16 that is longitudinally extending, and second housing piece 15 defines a second cavity 19 that is also longitudinally extending. A medicament cartridge 30 is positioned within second cavity 19, and includes a cartridge body 32 that contains a medicament 36. In an implementation, medicament 36 includes liquid epinephrine, however, in other embodiments different medicament compositions might be used, such as insulin, antibiotics, vaccines, antivenin, to name a few examples. Moreover, it should be appreciated that rather than a liquid medicament, a different injectable medicament such as a gel, an emulsion, or a mixable plural component liquid-liquid composition or liquid-solid composition might be used. The present disclosure should be understood without limitation to any particular medicament contained within medicament cartridge 30, or limitation as to the structure of medicament cartridge 30 itself. Embodiments are contemplated where a medicament cartridge 32 includes two chambers that are selectively fluidly connected upon activation of autoinjector 10, for example. Medicament cartridge 30 further includes an injection needle 34 that extends from cartridge body 32 in a direction of distal end 18. A stopper 40 may be positioned within second housing piece 15 and receives a sharp tip (not numbered) or the like of injection needle 34. In the illustrated embodiment, a barrier sack or envelope 410 extends about medicament cartridge 30 and stopper 40. It is contemplated that envelope 410 can completely encapsulate these components to maintain medicament to be injected as well as needle 34 in a sterile state. Materials of which envelope 410 can be made are known, including gas-permeable materials that enable gas sterilization. Needle 34 can puncture through envelope 410 when autoinjector 10 is activated. Also shown in FIG. 4 is another barrier or covering 400 that blocks cavity 19 to prevent contamination. Covering 400 could be a disk or the like glued to housing piece 15 or attached by any other suitable means, and punctured or otherwise ruptured or displaced by plunger 48 (discussed below) when autoinjector 10 is activated. Barrier 400 might be formed from a textile, a plastic film, a foil, or any of a variety of other materials and could be gas-permeable or potentially could fluidly seal cavity 19. Envelope 410 and barrier 400 might be used together in the same autoinjector, independent of one another, or not at all.

A hole 42 or injection port can be formed in second housing piece 15 at distal housing end 18, through which injection needle 34 is extended when autoinjector 10 is activated. A plug 38 is also coupled with cartridge body 32 and can be acted upon to both urge medicament cartridge 30 through second housing piece 15 and toward distal housing end 18, and to push medicament 36 through injection needle 34 for injecting into tissues of a user. Those skilled in the art will be familiar with the conventional intramuscular or subcutaneous injections whereby autoinjectors typically deliver medicament into the body of a user.

Autoinjector 10 further includes an actuator 44 positioned within first cavity 16, and having a force-producing device 46, and a plunger 48 coupled with force-producing device 46. It can be seen that actuator 44 is housed in an entirety or substantially in an entirety within first housing piece 13. Force-producing device 46 is adjustable from an energized state to a deployed state, to advance plunger 48 from first cavity 16 into second cavity 19 and urge medicament cartridge 30 in a distal direction through elongate housing 11 to extend injection needle 34 past distal housing end 18, when autoinjector 10 is in the use configuration. In an implementation, force-producing device 46 includes a coil spring held in compression between plunger 48 and first housing piece 13. It can be seen that plunger 48 can include a radially outwardly extending structure, generally in the nature of a flange (not numbered), and first housing piece 13 can also include an inwardly extending structure, which can also be a flange, with force-producing device 46 compressed between those structures. Those skilled in the art will contemplate many variations on the design set forth in the embodiment shown in FIG. 4 whereby a coil spring could be held in a compressed state, a tensioned or lengthened state, or other otherwise a mechanical spring device used to produce force for driving a plunger. In still other embodiments, a stored gas spring or the like could be used, to apply pneumatic or potentially even hydraulic force to plunger 48 for purposes set forth herein. In such a design, the gas spring could drive a piston that serves as the plunger or is coupled with the plunger, or could drive intermediate components to otherwise produce a linear, axial driving force.

Autoinjector 10 further includes a trigger 50 that can be used to fire autoinjector 10. Trigger 50 includes a trigger button body 150 that is coupled with or positioned about a deformable body 52 that is coupled to plunger 48. Deformable body 52 can be directly attached to plunger 48 and formed integrally therewith, or formed as a separate piece. A bore 54 extends through deformable body 52, and one or more slots 56 are formed longitudinally along deformable body 52 and are in communication with bore 54. In an implementation, slots 56 enable or enhance deformability of deformable body 52 under conditions suitable for firing autoinjector 10. It can further be seen that deformable body 52 extends through an opening 62 defined by first housing piece 13, and is positioned such that one or more retention tabs projecting in radially outward directions inhibit deformable body 52 being passed in a distal direction through opening 62. It will be recalled that force-producing device 46 may be in a compressed state, giving plunger 48 a tendency to advance through first housing piece 13 in a distal direction. Engagement of retention tab(s) 60 against first housing piece 13 inhibits passing deformable body 52 through opening 62, and thereby also inhibits advancing plunger 48. At the state shown in FIG. 4, safety 20 is coupled with trigger 50 and includes a pin 58 that extends through bore 54, to limit deformation of deformable body 52. In this embodiment, safety 20 can be understood as a removable insert positioned at least partially within deformable body 52 and limiting deformation thereof. Safety 20 is coupled with first housing piece 13 and caps proximal housing end 14. When safety 20 is decoupled from first housing piece 13, trigger 50 can be actuated to activate (fire) autoinjector 10, as further discussed herein.

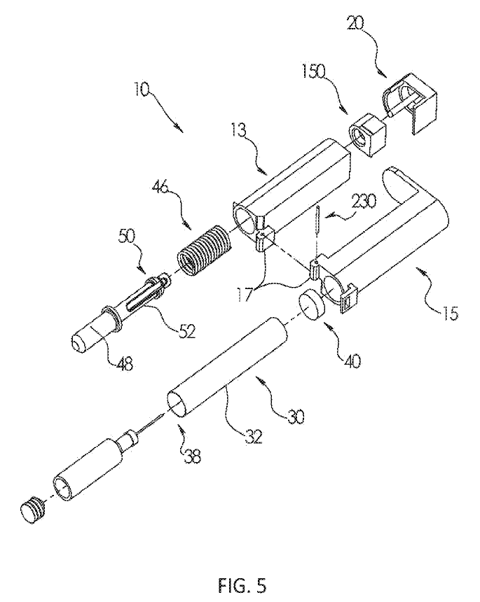

Referring also to FIG. 5, there is shown a disassembled view of certain of the components of autoinjector 10, and illustrating additional details. A hinge pin 230 is shown as it might appear passed through a bore in hinge 17. In other embodiments, a living hinge or the like that does not include or require a hinge pin might be used. It should be appreciated that the present disclosure is to be understood without limitation to any particular hinge device, components, or hinge configuration. In other instances, rather than a transverse hinge, a twisting motion or diagonal motion might be used to adjust a hinge to transition foldable body pieces between a use configuration and a storage configuration. Referring to FIG. 9, there are shown some additional features relating to trigger 50, and illustrating an angled inner surface(s) 154 of trigger button body 150, with trigger button body 150 decoupled from elongate deformable body 52. Surface 154 could have a conical shape in some embodiments. FIG. 9 also illustrates tab 60, and angled or sloping surfaces 64 on tabs 60. At the state depicted in FIG. 9, trigger button body 150 has been decoupled from its position of engagement about deformable body 52, however, it will be appreciated that when trigger button body 150 is positioned about deformable body 52, and axially advanced to engage surface 154 to bear against surfaces 64, tab 60 will move inwardly with the flexing of deformable body 52 to effectively reduce an outer diameter dimension of deformable body 52 and allow deformable body 52 to slip through opening 62. In this general manner, depressing trigger button body 150 with one's thumb when safety 20 is removed actuates autoinjector 10.

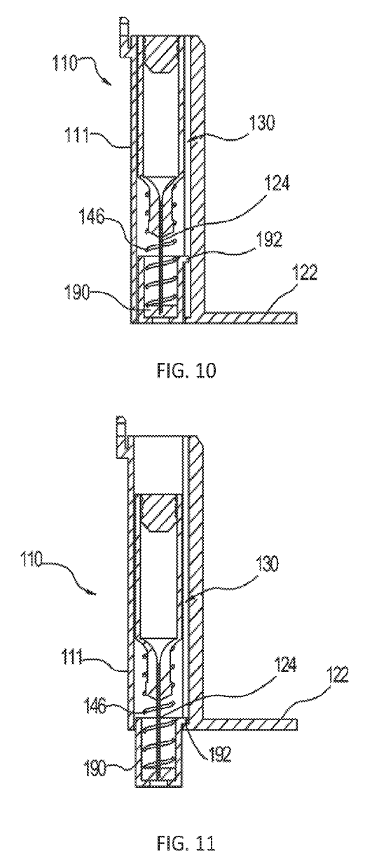

Referring now to FIG. 10, there is shown an autoinjector 110 according to another embodiment, and including an elongate housing 111, only one piece of which is shown, and having therein a medicament cartridge 130 and also a needle cover 190. It will be appreciated that the use of syringes, autoinjectors, and the like often requires the advancement of the injection needle out of the device, potentially creating a finger stick risk or other problems if not protected. A finger guard 122 generally extends radially outwardly in a manner analogous to the previously described embodiment. Referring also to FIG. 11, there is shown needle guard 190 advanced out of elongate housing 111, under the influence of an actuator 146 in the nature of a coil spring. It can be seen that actuator 146 can be compressed by the travel of medicament cartridge 130 through elongate housing 111, but needle guard 190 will be maintained within elongate housing 111 so long as autoinjector 110 is held in contact with a user's body. When autoinjector 110 is withdrawn, actuator 146 can urge needle guard 190 outwardly to surround injection needle 124. A projecting tab, flange, or other protuberance 192 upon needle guard 190 can limit outward travel of needle guard 190 under the influence of actuator 146.

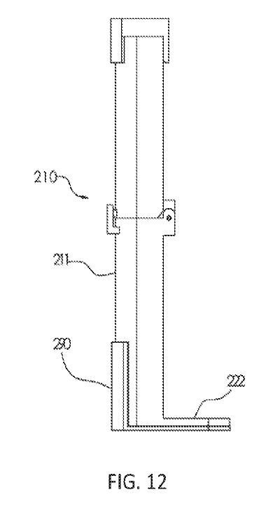

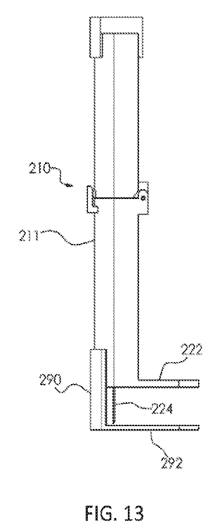

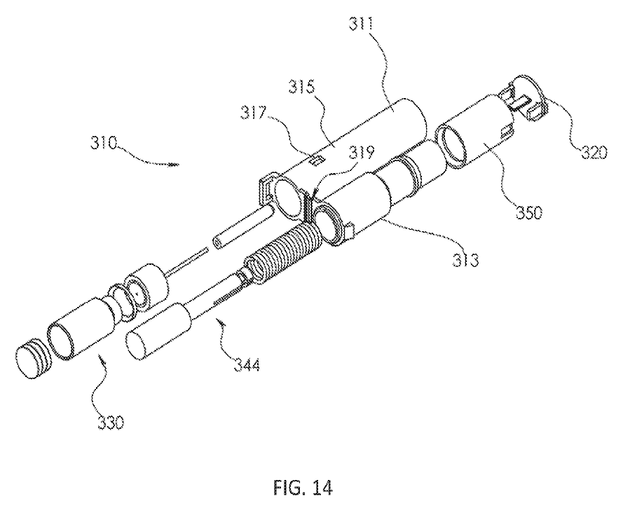

Referring also now to FIG. 12, there is shown an autoinjector 210 according to yet another embodiment, and including an elongate housing 211, a finger guard 222, and a needle guard 290. Referring now to FIG. 13, there is shown autoinjector 210 where needle guard 290 has been extended outwardly, under the influence of an actuator within elongate housing 211 and not visible in FIG. 13, to shield an injection needle 224. It can be seen that an outward projection 292 generally matched in length, and potentially matched in axial projection footprint, with finger guard 222 is shown.

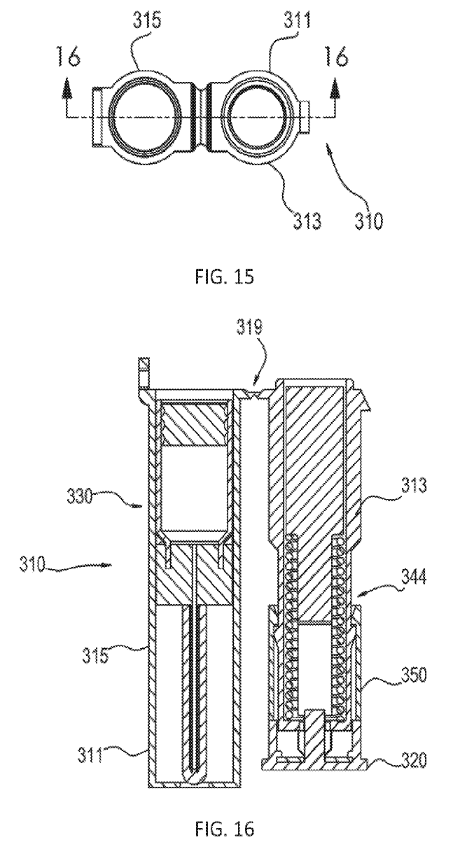

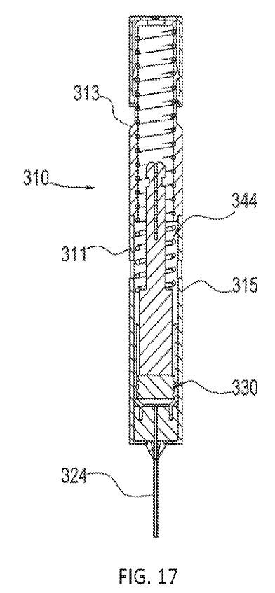

FIG. 14 illustrates an autoinjector 310 according to yet another embodiment, and including an elongate housing 311 having a first housing piece 313 and a second housing piece 315, an actuator 344, a safety 320, and a medicament cartridge 330. A view port 317 provides a window into elongate housing 311 to enable a user to visually inspect a medicament cartridge for signs of damage or degradation, such as cloudiness, solids, discoloration, or other problems. Autoinjector 310 has many similarities to other autoinjectors previously discussed herein, but is formed without a finger guard, and includes somewhat different shapes to the various housing components. It can also be noted that first housing piece 313 is formed as two separate individual pieces. A trigger button body is shown at 350. FIG. 15 illustrates an end view of autoinjector 310 as it might appear in its storage configuration, where the shape and surface contours of first housing piece 313 and second housing piece 315 are readily apparent. FIG. 16 shows a sectioned view taking along line 16-16 of FIG. 15, and illustrating components shown in FIG. 14 as they might appear with autoinjector 310 assembled, placed in its storage configuration and ready for service. It can also be noted as suggested above that autoinjector 310 includes an embodiment where a living hinge or the like 319 couples together the two body pieces. A viewing window or the like is shown at 317.

### INDUSTRIAL APPLICABILITY

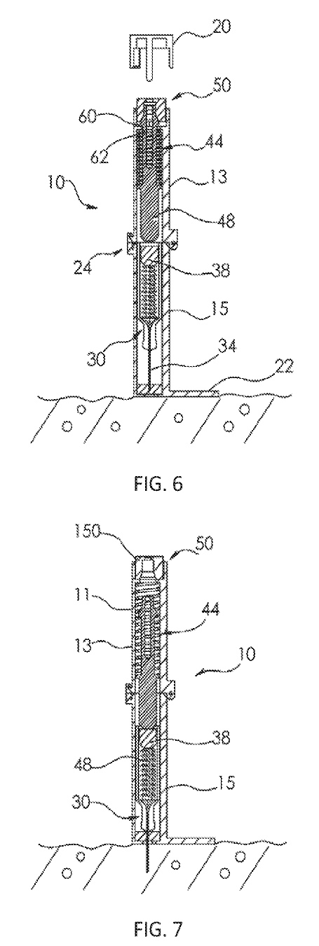

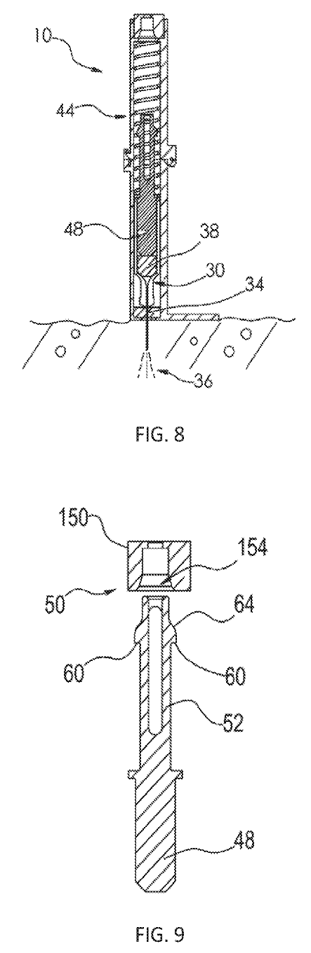

Referring now to the drawings generally, but in particular to FIG. 6, there is shown autoinjector 10 as it might appear positioned against the body of a user, and where safety 20 has been decoupled from first housing piece 13. At the state depicted in FIG. 6, actuator 44 is energized, and plunger 48 is within first housing piece 13 and medicament cartridge 30 within second housing piece 15. Latch 24 is latched, and finger guard 22 is positioned against the body of the user. Referring to FIG. 7, there is shown autoinjector 10 as it might appear where trigger button body 150 has been pushed to activate or fire trigger 50 by enabling deformable body 52 to advance through elongate housing 11 to drive plunger 48 against medicament cartridge 30. In particular, plunger 48 has contacted plug 38 and drives medicament cartridge 30 through second housing piece 15 to advance injection needle 34 into the body of the user. Referring now to FIG. 8, there is shown plunger 48 further advanced to drive plug 38 through medicament cartridge 30 and push medicament 36 through injection needle 34 and into the body of the patient.

The description of any one of the embodiments herein, including description of individual components and functioning, can be understood to refer analogously to all of the embodiments of the present disclosure except where otherwise indicated or apparent from the context. From the foregoing description, various applications and advantages of the present disclosure will be apparent. For instance, it will be appreciated that safety 20 is structured to adjust between a locking state, at which safety 20 inhibits firing actuator 44, and a release state, at which actuator 44 can be fired. Safety 20 is positionable in a locking state in each of the use configuration and the storage configuration of autoinjector 10. One can readily contemplate the difficulty in manipulating an autoinjector under conditions of duress, and the possibility of mis-firing or early firing an autoinjector, so that the medicament cannot be fully delivered or cannot be delivered at all, with potentially disastrous consequences. Because safety 20 remains engaged and inhibits firing actuator 44 even when autoinjector 10 is in the use configuration, these risks are reduced. Moreover, the positioning of finger guard 22 in a manner to trap safety 20 as described herein also reduces likelihood of mis-firing or dry firing of autoinjector 10 and the other autoinjectors contemplated within the scope of the present disclosure.

The present description is for illustrative purposes only, and should not be construed to narrow the breadth of the present disclosure in any way. Thus, those skilled in the art will appreciate that various modifications might be made to the presently disclosed embodiments without departing from the full and fair scope and spirit of the present disclosure. Other aspects, features and advantages will be apparent upon an examination of the attached drawings and appended claims. As used herein, the articles “a” and “an” are intended to include one or more items, and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. An autoinjector comprising:
an elongate housing including a first housing piece forming a proximal housing end and defining a first cavity, and a second housing piece forming a distal housing end and defining a second cavity;
a hinge coupling together the first housing piece and the second housing piece, and the first housing piece and the second housing piece being rotatable about the hinge to fold the elongate housing between a use configuration where the first cavity and the second cavity are in communication, and a storage configuration;
a medicament cartridge positioned within the second cavity, and including a cartridge body, and an injection needle extending from the cartridge body in a direction of the distal housing end; and
an actuator positioned within the first cavity, and including a force-producing device, and a plunger coupled with the force-producing device;
the force-producing device being adjustable from an energized state to a deployed state, to advance the plunger from the first cavity into the second cavity and urge the medicament cartridge in a distal direction to extend the injection needle past the distal housing end.

2. The autoinjector of claim 1 further comprising a trigger coupled with the actuator.

3. The autoinjector of claim 2 wherein the force-producing device includes a coil spring.

4. The autoinjector of claim 2 further comprising a safety inhibiting firing the actuator.

5. The autoinjector of claim 4 wherein the trigger includes a deformable body coupled with the plunger and having at least one retention tab.

6. The autoinjector of claim 5 wherein the safety includes a removable insert positioned at least partially within the deformable body and limiting deformation thereof.

7. The autoinjector of claim 4 wherein the safety is coupled with the first housing piece and caps the proximal housing end.

8. The autoinjector of claim 7 further comprising a finger guard extending radially outward from the distal housing end and holding the safety captive between the proximal housing end and the finger guard in the storage configuration.

9. The autoinjector of claim 1 further comprising a finger guard extending radially outward from the distal housing end.

10. An autoinjector comprising:
an elongate housing including a plurality of housing pieces;
a hinge coupling together two of the housing pieces, and the two of the housing pieces being rotatable about the hinge to fold the autoinjector between a use configuration, and a storage configuration;
a medicament cartridge positioned within the elongate housing and including an injection needle;
an actuator for deploying the injection needle, positioned within the elongate housing; and
a safety coupled with the actuator, and adjustable between a locking state at which the safety inhibits firing the actuator, and a release state, and the safety being positionable in the locking state in each of the use configuration and the storage configuration of the autoinjector.

11. The autoinjector of claim 10 further comprising a trigger coupled with the actuator and including at least one retention tab in contact with the elongate housing.

12. The autoinjector of claim 11 wherein the trigger includes a deformable body, and the safety includes an insert positioned within the deformable body and limiting deformation thereof.

13. The autoinjector of claim 10 wherein the safety is coupled with one of the plurality of housing pieces and caps an end of the one of the plurality of housing pieces.

14. The autoinjector of claim 12 further comprising a finger guard extending radially outward from the elongate housing end and holding the safety captive between the end of the elongate housing and the finger guard in the storage configuration.

15. The autoinjector of claim 10 further comprising a needle cover movable between a retracted position and an advanced position to shield the injection needle when extended out of the elongate housing.

16. The autoinjector of claim 10 further comprising a latch attached to at least one of the plurality of housing pieces and structured to latch the elongate housing in the use configuration.

17. An autoinjector assembly comprising:
an autoinjector including an elongate housing having a first housing piece, and a second housing piece, and a hinge coupling together the first housing piece and the second housing piece, and the first housing piece and the second housing piece being rotatable about the hinge to fold the elongate housing between a use configuration, and a storage configuration;
the autoinjector further including a medicament cartridge including an injection needle, and an actuator for advancing the medicament cartridge through the elongate housing to extend the injection needle out of the elongate housing; and
a carrying accessory including a container having a cavity formed therein and receiving the autoinjector in the storage configuration, and a strap attached to the container and structured for wearing of the carrying accessory by a user.

18. The assembly of claim 17 wherein a first cavity is formed in the first housing piece and a second cavity is formed in the second housing piece, and wherein the medicament cartridge and the actuator are positioned within the first cavity and the second cavity, respectively.

19. The assembly of claim 18 wherein the elongate housing includes a proximal housing end formed by the first housing piece, and a distal housing end formed by the second housing piece, and wherein the autoinjector further includes a safety coupled with the proximal housing end and inhibiting firing the actuator.

20. The assembly of claim 17 wherein the autoinjector has a substantially rectangular shape in the storage configuration, and the cavity has a substantially rectangular shape congruent with the substantially rectangular shape of the autoinjector.
